(19) United States
(12) Patent Application Publication
(10) Pub. No.: US 2009/0023693 A1
(43) Pub. Date: Jan. 22, 2009

(54) NEW DROSPIRENONE/17BETA-ESTRADIOL REGIMEN, PHARMACEUTICAL COMBINATION PRODUCT AND KIT FOR PERFORMING THIS REGIMEN

(76) Inventors: Vladimir HANES, Tarrytown, NY (US); Hartnut Blode, Berlin (DE); Rolf Schurmann, Teltow (DE); Bernd Dusterberg, Oberkramer/ OT Barenklau (DE); Joachim Marr, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/098,831

(22) Filed: Apr. 7, 2008

Related U.S. Application Data
(60) Provisional application No. 60/910,326, filed on Apr. 5, 2007.
(60) Provisional application No. 61/035,285, filed on Mar. 10, 2008.
(60) Provisional application No. 61/040,494, filed on Mar. 28, 2008.

(51) Int. Cl. A61K 31/56; A61P 15/18

(52) U.S. Cl. 514/170

(57) ABSTRACT

The present invention relates to a pharmaceutical combination product with at least 21 daily consecutive dosage units containing from 2.0 mg to 3.0 mg of drospirenone and 1.0 to 2.0 mg of 17β-estradiol in each daily dosage unit followed by intermittent daily dosage units containing the same or smaller amount of drospirenone (i.e. 0.5 mg to 3.0 mg) as the consecutive daily dosage units wherein each intermittent daily dosage unit is preceded by at least one day without administration of drospirenone.

These pharmaceutical combination products can be used for female oral contraception, guarantee a withdrawal bleeding each 4 weeks and allow for the full maintenance of the drospirenone related benefits.

This application claims the benefit of the filing date of U.S. Provisional Application Ser. No. 60/910,326 filed Apr. 5, 2007, U.S. Provisional Application Ser. No. 61/035,285 filed Mar. 10, 2008 and U.S. Provisional Application Ser. No. 61/040,494 filed Mar. 28, 2008.

The present invention relates to a new regimen for administration of a pharmaceutical composition containing Drospirenone (DRSP) and 17β-Estradiol (E2) to human females for Contraception as well as for Contraception and Hormone Therapy in Perimenopausal Women.

Drospirenone containing OCs (Oral Contraceptives) already available are the products Yasmin and Yaz.

For Hormone Therapy the product Angeliq containing Drospirenone and 17β-Estradiol has been developed.

Standard contraceptive pills are administered in 28-day cycles, utilizing usually 21 days of active pills containing progestin plus estrogen, followed by a 7 days period of hormone free or inactive pills (21+7 regimen). The administration of active pills has recently been extended to 24 days with only 4 hormone free days (24+4 regimen). Also, extended regimens have been developed with continuous administration of active pills for up to three months (84+7 regimen). The extended regimens are an option for women who wish to reduce the frequency of withdrawal bleeding for convenience or due to symptoms and complaints associated with menstruation and hormone withdrawal.

All these regimens have a hormone free period which has the aim to trigger a withdrawal bleeding. However, such ‘hormone free period’ does not really exist in physiologic conditions in women and it is, in fact, completely artificial. Also, a ‘hormone free period’ does not have rationale when a therapeutic effect of estrogen and/or progestin is expected, e.g., relieve of vasomotor symptoms in peri- and postmenopausal women by estrogen; BP lowering effects in prehypertensive (systolic office cuff blood pressure 120-139 mmHg or diastolic office cuff blood pressure 80-89 mmHg) and hypertensive (systolic office cuff blood pressure >=140 mmHg or diastolic office cuff blood pressure >=90 mmHg) women and potassium-sparing effects based on anti-aldosterone properties of drospirenone.

Drospirenone has pharmacodynamic properties very similar to those of progesterone and differs from the classic progestins in its derivation from spirolactone. The major effect of drospirenone besides its progestational activity is its anti-aldosterone activity. Based on these properties of drospirenone, a reduced salt and water retention was observed and blood pressure was lowered in hypertensive women. The affinity of drospirenone for the mineralocorticoid receptor is about five times that of aldosterone, the naturally occurring mineralocorticoid. Drospirenone has been developed for contraception in combination with ethinyl estradiol (EE) in fertile women (daily administration of 3 mg DRSP combined with 20 or 30 μg EE, 21-day and 24-day regimens). Also, several continuous combinations of drospirenone with 17-β estradiol have been developed for the hormone therapy of postmenopausal women.

Perimenopause marks the interval in which a woman's body begins its transition into menopause. The perimenopause encompasses the years leading up to menopause—anywhere from two to eight years—plus the first year after the final menstruation. During this time, function of the ovaries declines and the body's estrogen levels drop. For most women, this takes place between ages 35 and 50 years. Most perimenopausal women experience changes in their menstrual cycle. When estrogen levels begin to drop, the follicular phase of the cycle may be shortened, and this can shorten the total cycle from 28-30 days to 24-26 days, resulting in more frequent periods. On the other hand, some women begin having longer cycles because they are not ovulating as frequently. These changes can be quite different on an individual basis. Additionally, this declining/fluctuating estrogen level can produce a host of disturbing symptoms: hot flashes, increasing vaginal dryness, sleep problems, mood swings, PMS-like symptoms, decreased sex drive, breast tenderness and many other signs and symptoms.

It is a first object of the present invention to provide a regimen which allows at the same time making use of the drospirenone related benefits throughout the woman's complete menstrual cycle and/or over the complete administration period whereby on the other side good cycle control (i.e. an acceptable bleeding pattern) and especially reliable introduction of (artificial) withdrawal bleeding shall be guaranteed.

It is another object of the invention to provide a new drospirenone/17-β estradiol (DRSP/E2) pill regimen which may be used as an OC in younger women and which is also intended to be used in the above mentioned population of the perimenopausal women to provide the still necessary contraception and treatment of menopausal symptoms and cycle control/irregular bleeding which might already be required in this stage of life.

Such product will combine the natural estrogen E2 and the synthetic progestin DRSP, which is closely related to the natural progestin progesteronein its pharmacological profile but which is effectively bioavailable via the oral route in contrast to progesterone.

EP 0 253 607 already discloses the use of a composition comprising an estrogen selected from
0.075-1.50 mg of 17β-estradiol,
0.012-0.025 mg of ethinyl estradiol, and
0.025-0.050 mg of mestranol;
   and a progestogen selected from
0.035-0.085 mg of levonorgestrel,
0.015-0.060 mg of gestodene,
0.035-0.085 mg of desogestrel,
0.035-0.085 mg of 3-ketodesogestrel, and
0.10-0.30 mg of norethindrone
   for the manufacture of a dosage form for providing hormonal replacement therapy and contraception for a pre-menopausal woman by administration of the dosage form for 23 to 26 days, beginning at day one of the menstrual cycle, followed by 2 to 5 pill-free or blank pill days, for a total of 28 days in the administration cycle.

This composition is not intended to be used as a contraceptive in younger women. Also drospirenone is not mentioned as a possible progestogenis component.

The 24+4 regimen mentioned above is described i.a. in PCT/EP94/04274 and US RE37,564 E. Claim 1 of this patent reads on
a combination product for oral contraception, comprising (a) 23 or 24 dosage units, each containing an estrogen selected from >2.0 to 6.0 mg of 17β-estradiol and 0.02 mg of ethinylestradiol; and a gestagen selected from 2.5 to 3.0 mg of drospirenone and 1 to 2 mg of cyproterone acetate, and (b) 5 or 4, respectively, active ingredient-free placebo pills or other indications to show that the daily administration of the 23 or 24 dosage units respectively, is to be followed by 5 or 4, respectively pill-free or placebo pill days.

The objects of the present invention are achieved in its broadest sense by a pharmaceutical combination product with at least 21 daily consecutive dosage units containing from 2.0 mg to 3.0 mg of drospirenone and 1.0 to 2.0 mg of 17β-estradiol or 10 to 20 μg of 17α-ethinyl estradiol in each daily dosage unit followed by intermittent daily dosage units containing the same or smaller amount of drospirenone as the consecutive daily dosage units wherein each intermittent daily dosage unit is preceded by at least one day without administration of drospirenone.

Intermittent daily dosage unit means a dosage unit the administration of which does not follow directly, i.e. on the next day, the administration of the previous dosage unit. The first intermittent daily dosage unit is separated from the last dosage unit of the consecutive daily dosage units by at least one day without administration of a hormone. Between the first and the second intermittent daily dosage units (and if the case may be also between the next intermittent daily dosage units) also at least one day without administration of a hormone is provided.

According to a preferred embodiment of the invention placebo pills are included for the days without administration of a hormone.

In case of another embodiment of the invention (vide infra) where a tetrahydrofolate may be present in the pharmaceutical combination product of the invention the this tertahydrofolate, preferably Metafolin, is present in the dosage units which precede the intermittent daily dosage units.

The present invention also relates to a kit containing the above described combination product.

In one embodiment of the invention each intermittent daily dosage unit is preceded by one day without administration of drospirenone.

In another embodiment of the invention at least one intermittent daily dosage unit is preceded by two days without administration of drospirenone.

In yet another embodiment of the invention one intermittent daily dosage unit is preceded by at least one day without administration of drospirenone.

In a further embodiment according to the invention the regimen provides for 23 daily oral dosage units and for intermittent daily dosage units to be administered on days 25 and 27 of the 28 days menstrual cycle.

Yet another embodiment of the invention provides for 24 daily oral dosage units and for intermittent daily dosage units to be administered on days 26 and 28 of the 28 days menstrual cycle.

In an even further embodiment according to the invention the regimen provides for 24 daily oral dosage units and for an intermittent daily dosage unit to be administered on day 27 of the 28 days menstrual cycle.

The new regimens according to the invention contains from 2.0 mg to 3.0 mg of drospirenone and 1.0 to 2.0 mg, preferably 1.50 mg of 17β-estradiol or 10 to 20 μg ethinylestradiol per daily dosage unit during the uninterrupted daily administration for at least 21 days and the same or smaller amount of drospirenone in the dosage units which are administered intermittently

each second day thereafter or
the third day thereafter
or the first intermittent dosage unit on the third day thereafter and the second intermittent dosage unit on the second day after the first intermittent dosage unit or the first intermittent dosage unit on the second day thereafter and the second intermittent dosage unit on the third day after the first intermittent dosage unit to complete the 28 days cycle.

According to the present invention it has been found that such new dosage regimen surprisingly ensures reliable induction of the withdrawal bleeding before uninterrupted daily administration of the DRSP/E2-administration commences again. This is surprising since some drospirenone is administered throughout the otherwise hormone free period. The “off/on” phase (days 22-28, preferably 25-28) is deemed to increase ovarian suppression with reliable induction of the withdrawal bleeding in the presence of a progestin in the otherwise hormone-free (pill-free) interval.

The new regimens provide an acceptable bleeding profile with respect to parameters as total number of bleeding days, intensity of bleeding, lengths of withdrawal bleeding, etc. At the same time such new regimens with an intermittent administration of the same or reduced amount of drospirenone “in the break” guarantees full maintenance of the drospirenone benefits throughout the whole duration of the administration without intermittent decrease or interruption of the drospirenone specific benefits.

The administered E2 dosages are sufficient to maintain normal physiological bone mineral density. Replacement of ethinylestradiol by E2 is expected to provide significant benefits. One thereof is less impact on metabolic parameters, such as liver protein biosynthesis.

In another aspect of the invention 10 to 20 μg, preferably 15 μg of 17-ethinyl estradiol are contained as an estrogen per daily dosage unit.

The parts of the regimens and pharmaceutical combinations according to the present invention which are constituted by the at least 21 daily consecutive dosage units may be monophasic, i.e. in each dosage unit thereof the same amounts of 17β-estradiol and drospirenone are contained or these parts may be multiphasic, i.e. the amounts of 17β-estradiol and/or drospirenone may be changed stepwise.

In one embodiment of a regimen and pharmaceutical composition according to the invention the amount of 17β-estradiol increases stepwise from 1.0 mg of 17β-estradiol in the first step to 1.5 mg of 17β-estradiol in the second step to 2.0 mg of 17β-estradiol in the third step. The amount of drospirenone in each consecutive dosage unit remains constant. 3.0 mg of drospirenone are preferred.

In case of 24 consecutive daily dosage units each step has 6 to 10 and preferably 8 daily dosage units.

Thereby constant drospirenone amounts of 3.0 mg per each consecutive dosage unit are combined with increasing 17β-estradiol doses to obtain high ovarian suppression (comparable to YAZ) and to counteract down-regulation of estradiol receptors during the treatment cycle.

Another embodiment provides for stepwise increase of the 17β-estradiol and stepwise decrease of the drospirenone amount, starting with 1.0 mg of 17β-estradiol to 1.5 mg of 17β-estradiol to 2.0 mg of 17β-estradiol whereas in the same sequence the drospirenone amount decreases from 3.0 mg of drospirenone to 2.5 mg of drospirenone to 2.0 mg of drospirenone.

Again in case of 24 consecutive daily dosage units each step has 6 to 10 and preferably 8 daily dosage units.

A daily dosage amount of 3.0 mg of drospirenone per dosage unit is preferred in case of monophasic 17β-estradiol and drospirenone.

In another embodiment the intermittent dosage units contain less drospirenone than the daily dosage units in the continuos and uninterrupted part (day 1 to at least day 21) of the regimen. In this embodiment for instance 1.0 mg drospirenone are contained in the intermittent dosage units.

In even a further embodiment of the invention a tetrahydrofolate is contained in each daily dosage unit in addition to the estrogen and drospirenone, in the intermittend dosage units in addition to the estrogen as well as in the remaining daily units without any drospirenone. Pharmaceutical compositions containing an estrogen and/or a progestin as well as 5-methyl-(6S)-tetrahydrofolate are described in WO 2006/120035 which is incorporated herein by reference.

WO 2006/120035 discloses oral contraceptives which, although able to prevent diseases caused by folate deficiency, at the same time are unable to mask the symptoms of vitamin B₁₂ deficiency. The respective administration regime ensures that the consumer of the pharmaceutical composition of that invention is reliably protected also for a certain time after discontinuation from disorders or malformations caused by folate deficiency, in particular from neural tube defects. Both these also apply in the case of a homozygous or heterozygous polymorphism of methylenetetrahydrofolate reductase in the user, which adversely affects the utilizability of folic acid by the body and thus its biological activity to prevent neural tube defects.

The addition of a 5-methyl-(6S)-tetrahydrofolate to the pharmaceutical combination product of the present invention serves the same purpose as it does in WO 2006/120035.

Reference to 5-methyl-(6S)-tetrahydrofolates in the form according to the present invention means the free acid form and pharmaceutically acceptable salts and modifications of 5-methyl-(6S)-tetrahydrofolic acid (N-[4-[[(2-amino-1,4,5,6,7,8-hexahydro-4-oxo-5-methyl-(6S)-pteridinyl)methyl]amino]benzoyl]-L-glutamic acid).

Pharmaceutically acceptable salts are intended to be both pharmacologically and pharmaceutically acceptable. Such pharmacologically and pharmaceutically acceptable salts may be alkali metal or alkaline earth metal salts, preferably sodium, potassium, magnesium or calcium salts. The calcium salt is particularly preferred.

The amount used for example of the calcium salt, which is particularly preferred according to the invention, of 5-methyl-(6S)-tetrahydrofolic acid (metafolin) is between 0.1 and 10 mg, preferably 0.4 to 1 mg, particularly preferred 451 μg (equivalent to 400 μg of folic acid or 416 μg of 5-methyl-(6S)-tetrahydrofolic acid (metafolin)).

Crystalline modifications disclosed in EP 1044975 are preferably employed as modifications of 5-methyl-(6S)-tetrahydrofolates.

By way of example two preferred regimens are illustrated in the scheme below (the inclusion of Metafolin into the regimens is optional):

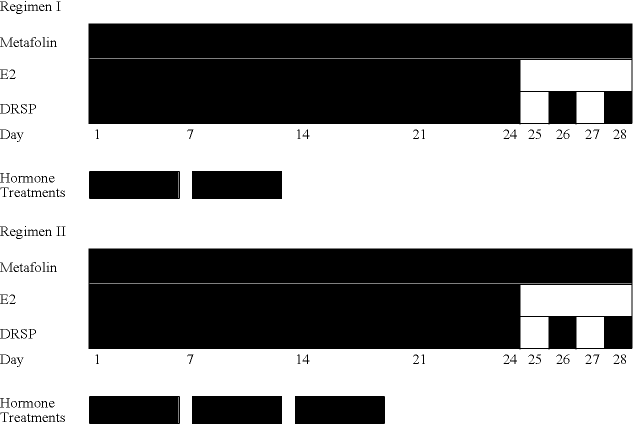

The present invention also refers to pharmaceutical combination product to perform the above mentioned regimens.

In its broadest aspect the invention provides a pharmaceutical combination product with at least 21 daily consecutive dosage units containing from 2.0 mg to 3.0 mg of drospirenone and 1.0 to 2.0 mg, preferably 1.50 mg of 17β-estradiol in each daily dosage unit followed by intermittent daily dosage units containing the same or smaller amount of drospirenone as the consecutive daily dosage units wherein each intermittent daily dosage unit is preceded by one day without administration of drospirenone.

Instead of 1.0 to 2.0 mg, preferably 1.50 mg of 17β-estradiol according to the invention 10 to 20 μg, preferably 15 μg of 17α-ethinyl estradiol are contained as an estrogen per daily dosage unit.

Placebo tablets may be introduced in the regimens on days with no hormone intake (i.e., days 25 and 27) with the aim to increase women's compliance and not to forget to take a pill every day.

According to an embodiment of the invention in case that the pharmaceutical combination also contains a 5-methyl-(6S)-tetrahydrofolate each hormone-free “placebo” contains this 5-methyl-(6S)-tetrahydrofolate, too and preferably in the same amount as the daily dosage units do.

Such new regimen and pharmaceutical combination result in rather continuous serum levels of drospirenone leading to higher contraceptive efficacy as compared to 21/7 or 24/4 regimens. The continuous serum levels of drospirenone are particularly important for a fail-safe ovulation inhibition effect of the combination when 17β-estradiol is used as the estrogen since it is much weaker then ethinylestradiol, the currently used estrogen in oral contraceptives.

Even further all the benefits known for drospirenone are maintained effectively throughout the complete administration period. These benefits in first instance are the therapeutic activity in treatment of PMDD (Premenstrual Dysphoric Disorders), acne and the ability of dospirenone to keep the body weight virtually unchanged due to its anti-mineralcorticoid effect counteracting the water retention by the estrogen.

Additional drospirenone benefits include lowering blood pressure in pre-hypertensive and hypertensive women, its ability to keep the bone mass density (BMD) constant in comparison to other 17α-estradiol containing preparations.

The effect of the regimens with respect to ovulation inhibition and acceptable withdrawal bleeding are tested in clinical studies. By these studies the ovulation inhibition effect of the new regimens is evaluated in one study, during which the bleeding pattern, cycle control, and tolerability of the regimens are also monitored.

A multi-center, double-blind, randomized, parallel-group study is conducted to evaluate cycle control and safety of different regimens of an oral contraceptive containing 17β-estradiol (E2) and drospirenone (DRSP) in healthy female volunteers aged between 18 and 35 years over 7 cycles.

The following 4 different treatment and dose regimens, treatment groups A to D, are evaluated. Approximately 100 volunteers are treated per each group. Route of administration is oral.

### Treatment A/mono DRSP 2×

### Day 1-24: 1.5 mg E2+3 mg DRSP

Day 25: placebo

### Day 26: 3 mg DRSP

Day 27: placebo

### Day 28: 3 mg DRSP

### Treatment B/mono DRSP 1×

### Day 1-24: 1.5 mg E2+3 mg DRSP

Day 25: placebo
Day 26: placebo

### Day 27: 3 mg DRSP

Day 28: placebo

### Treatment C/tri con DRSP 1×

### Day 1-8: 1 mg E2+3 mg DRSP

### Day 9-16: 1.5 mg E2+3 mg DRSP

### Day 17-24:2 mg E2+3 mg DRSP

Day 25: placebo
Day 26: placebo

### Day 27: 3 mg DRSP

Day 28: placebo

### Treatment D/tri dec DRSP 1×

### Day 1-8: 1 mg E2+3 mg DRSP

### Day 9-16: 1.5 mg E2+2.5 mg DRSP

Day17-24:2 mgE2+2 mgDRSP
Day 25: placebo
Day 26: placebo

### Day 27: 2 mg DRSP

Day 28: placebo

The volunteers (healthy female volunteers, age 18-35 years inclusive) are treated over 7 treatment cycles, each consisting of 28 days (total 196 days), one tablet per day

### Efficacy Variables 1

Primary efficacy variable
Number of intracyclic bleeding episodes (including spotting) in Cycles 2 to 7

### Secondary Efficacy Variables

Number of intracyclic bleeding days (including spotting) in Cycles 2 to 7
Number of withdrawal bleeding episodes in Cycles 1 to 6
Bleeding pattern
   Number of bleeding/spotting days
   Number of bleeding days (excluding spotting)
   Number of spotting-only days
   Number,(mean length, maximum length, and range of length) of bleeding/spotting episodes
   Number (mean length, maximum length, and range of length) of spotting-only episodes.
Cycle control
Withdrawal bleeding
   Number of volunteers with/without withdrawal bleeding
   Length of withdrawal bleeding episodes
   Maximum intensity of withdrawal bleeding episodes
   Onset of withdrawal bleeding episodes
Intracyclic bleeding (including spotting)
   Number of volunteers with/without intracyclic bleeding
   Number and maximum length of intracyclic bleeding episodes
   Number of intracyclic bleeding days
   Maximum intensity of intracyclic bleeding episodes
Intracyclic bleeding (excluding spotting)
   Number of volunteers with/without intracyclic bleeding
   Number and maximum length of intracyclic bleeding episodes
   Number of intracyclic bleeding days
Women with intracyclic bleeding (including spotting)
   Number of volunteers with at least one intracyclic bleeding episode in Cycles 2-6
   Number of volunteers with at least one intracyclic bleeding episode in Cycles 2-7
Women with intracyclic bleeding (excluding spotting)
   Number of volunteers with at least one intracyclic bleeding episode in Cycles 2-6
   Number of volunteers with at least one intracyclic bleeding episode in Cycles 2-7
Subjective assessment of treatment
   Safety variables:
Baseline findings and adverse events (AE)
Safety laboratory tests (incl. pregnancy tests)
Vital signs
Physical and gynecological examination (incl. breast palpation, transvaginal ultrasonography [TVU] and cytological cervical smear).

The regimens provide an acceptable bleeding profile and good tolerance.

The ovulation inhibition achieved by the regimens according to the present invention is evaluated in a randomized, double-blind clinical study. Approximately 50 volunteers are included within one treatment group. The study encompasses 1 pre-treatment and 3 treatment cycles. The primary clinical endpoint is to determine the number of volunteers with incomplete ovulation inhibition. Incomplete ovulation inhibition is defined by a Hoogland score 6 (ovulation) in treatment cycles 2 or 3. Successful ovulation inhibition is demonstrated if less than 5% of PPS (Per Protocol Set) show incomplete ovulation inhibition.

All regimens inhibit ovulation effectively.

Pharmaceutical combinations of the invention can be formulated according to accepted pharmaceutical practice, with a conventional pharmaceutically acceptable vehicle, carrier, excipient, binder, preservative, stabilizer, flavor, and/or adjuvant, etc, for any given type of unit dosage form.

Formulations for oral administration are conventional in the art. For example, tablets generally contain a pharmaceutically acceptable carrier, e.g., a binder such as gum tragacanth, acacia, corn starch or gelatin; an excipient such as dicalcium phosphate or celluose; a disintegrating agent such as corn starch or alginic acid; a lubricant, such as magnesium stearate; and/or a sweetening agent or flavoring agent. When the dosage unitform is a capsule, it may contain in addition to materials of the above type a liquid carrier such as a fatty oil. Various other materials may be present as coatings or to otherwise modify the physical form of the dosage unit. For instance, tablets or capsules may be coated with shellac, sugar or both. A syrup or elixir may contain the active compound, water, alcohol or the like as the carrier, glycerol assolubilized, sucrose as sweetening agent, methyl and propyl parabens as preservatives, a dye and a flavoring such as cherry or orange. When administered orally as a suspension, these compositions may contain microcrystalline cellulose for imparting bulk, alginic acid or sodium alginate as a suspending agent, methylcellulose as a viscosity enhancer, and sweetners/flavoring agents known in the art. As immediate release tablets, these compositions may contain microcrystalline cellulose, dicalcium phosphate, starch, magnesium stearate and lactose and/or other excipients, binders, disintegrants, diluents and lubricants known in the art.

Drospirenone can be obtained from commercial sources (e.g., from Bayer Schering Pharma AG) or can by synthesized by conventional methods, e.g., according to the methods disclosed in U.S. Pat. No. 6,121,465 and Drugs of the Future 2000, 25 (12), 1247-1256.

While it is clearly preferred according to the invention that the dosage units are adapted for oral administration and the stated daily dosages are given for the oral administration it is also in the ambit of the invention to administer the daily dosages by other routes known to be effective for hormonal contraception, e.g. via the transdermal or transmuccosal route.

If the dosage units are administered by non-oral routes adjustment of the daily doses might be necessary. For instance in case of transdermal administration 0.05 mg of transdermally administered E2 roughly translates into 1 mg of orally administered E2, i.e. E2 is about 20 times better available upon transdermal compared to oral administration.

The bioavailabilities of DRSP after oral and transdermal administration are roughly the same i.e. the doses of DRSP to be administered transdermally are roughly the same as those given in the present specification relating to oral administration.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of U.S. Provisional Application Ser. No. 60/910,326, filed Apr. 5, 2007, U.S. Provisional Application Ser. No. 61/035,285 filed Mar. 10, 2008 and 61/040,494 filed Mar. 28, 2008, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Pharmaceutical combination product with at least 21 daily consecutive dosage units containing from 2.0 mg to 3.0 mg of drospirenone and 1.0 to 2.0 mg of 17β-estradiol in each daily dosage unit followed by intermittent daily dosage units containing the same or smaller amount of drospirenone as the consecutive daily dosage units wherein each intermittent daily dosage unit is preceded by at least one day without administration of drospirenone.

2. Pharmaceutical combination product according to claim 1 wherein each intermittent daily dosage unit is preceded by one day without administration of drospirenone.

3. Pharmaceutical combination product according to claim 1 wherein at least one intermittent daily dosage unit is preceded by two days without administration of drospirenone.

4. Pharmaceutical combination product according to claim 1 wherein one intermittent daily dosage unit is preceded by at least one day without administration of drospirenone.

5. Pharmaceutical combination product according to claim 1 with 23 consecutive daily oral dosage units and two intermittent daily dosage units to be administered on days 25 and 27 of the 28 days menstrual cycle.

6. Pharmaceutical combination product according to claim 1 with 24 consecutive daily oral dosage units and two intermittent daily dosage units to be administered on days 26 and 28 of the 28 days menstrual cycle.

7. Pharmaceutical combination product according to claim 1 with 24 daily consecutive oral dosage units and an intermittent daily dosage unit to be administered on day 27 of the 28 days menstrual cycle.

8. Pharmaceutical combination product according to claim 1 containing from 2.0 mg to 3.0 mg of drospirenone and 1.0 to 2.0 mg, preferably 1.50 mg of 17β-estradiol per daily dosage unit during the uninterrupted daily administration for at least 21 days and the same or smaller amount of drospirenone in the dosage units which are administered intermittently.

9. Pharmaceutical combination product according to claim 1 containing 1.5 mg of 17β-estradiol per daily dosage unit.

10. Pharmaceutical combination product according to claim 1 with 23 daily consecutive dosage units.

11. Pharmaceutical combination product according to claim 1 with 24 daily consecutive dosage units.

12. Pharmaceutical combination product according to claim 1 wherein each consecutive dosage unit contains from 2.0 mg to 3.0 mg of drospirenone.

13. Pharmaceutical combination product according to claim 1 wherein each consecutive dosage unit contains 3.0 mg of drospirenone.

14. Pharmaceutical combination product according to claim 1 wherein each intermittent dosage unit contains less than 3.0 mg of drospirenone.

15. Pharmaceutical combination product according to claim 14 wherein each intermittent dosage unit contains 0.5 or 2.0 mg of drospirenone.

16. Pharmaceutical combination product according to claim 1 wherein instead of 1.0 to 2.0 mg of 17β-estradiol 10 to 20 μg of 17α-ethinylestradiol are contained as an estrogen per daily dosage unit.

17. Pharmaceutical combination product according to claim 1 in which a tetrahydrofolate is contained in each daily dosage unit in addition to the estrogen and drospirenone and in the intermittent dosage units in addition to drospirenone as well as in the remaining daily units without any drospirenone.

18. Pharmaceutical combination product according to claim 17 wherein 0.1 to 10 mg of Metafolin is contained in each dosage unit.

19. Pharmaceutical combination product according to claim 18 wherein 0.4 to 1.0 mg of Metafolin is contained in each dosage unit.

20. A pharmaceutical kit containing a pharmaceutical combination product according to claim 1.
